(19) United States
(12) Patent Application Publication
(10) Pub. No.: US 2015/0297266 A1
(43) Pub. Date: Oct. 22, 2015

(54) MODULAR MULTI-AXIAL SCREW SYSTEM

(71) Applicant: David Louis Kirschman, Dayton, OH (US)

(72) Inventor: David Louis Kirschman, Dayton, OH (US)

(73) Assignee: X-SPINE SYSTEMS, INC., Miamisburg, OH (US)

(21) Appl. No.: 14/678,129

(22) Filed: Apr. 3, 2015

Related U.S. Application Data
(60) Provisional application No. 61/982,043, filed on Apr. 21, 2014.

(51) Int. Cl. A61B 17/70

(52) U.S. Cl. CPC A61B 17/7032; A61B 17/7035

(57) ABSTRACT

A modular pedicle screw comprising a tulip or receiver having a body comprising a through bore for receiving a screw having a screw head, the tulip or receiver also comprising a rod receiving channel for receiving a rod, the body comprising a seat for receiving and supporting at least a portion of the screw head, a wall having at least one resilient or elastic arm, the at least one resilient or elastic arm having at least a portion that is operatively positioned in relation to the screw head to retain the screw head in the tulip or receiver.

### CROSS-REFERENCE TO RELATED APPLICATION

The present application claims priority to provisional U.S. Application Ser. No. 61/982,043 filed Apr. 21, 2014, to which Applicant claims the benefit of the earlier filing date. This provisional application is incorporated herein by reference and made a part hereof.

### BACKGROUND OF THE INVENTION

1. Field of the Invention

This invention relates to a modular multi-axial or polyaxial screw system.

2. Description of the Related Art

Multi-axial screw systems are a mainstay of spinal orthopedic procedures. Exemplary inventions such as those disclosed in U.S. Pat. Nos. 4,641,636; 4,653,481; 4,805,602; 4,946,458; 4,987,892; 5,005,562; 5,176,780; 5,261,909; 5,360,431; 5,443,467; 7,604,655; 7,662,172; 7,686,835; 7,717,943; 8,012,185; 8,016,866; 8,066,745; 8,092,504; 8,097,025; 8,142,481; 8,147,522; 8,382,806 and U.S. Publication Nos. 2007/0123867 and 2013/0150893, which comprise various rod-based fixation systems, in which a screw is coupled to a fixation rod. Such coupling is multiaxial in nature, in order to respect variable anatomical relationships between the implanted rod and screw position. In the prior art, designs typically comprise at a minimum, a tulip-shaped rod-locking component, a ball-end threaded screw shaft component, a rod component and a locking cap component.

With few exceptions, the extant systems are permanent assemblies permanently incorporating all the components. In fewer systems, exemplified by the Synthes Click-X system offered by Synthes Spine of West Chester, Pa., the tulip is mounted onto the ball as a separate intra-operative step. Such systems incorporate a captive split-ring mechanism at the base of the tulip to irreversibly capture the screw ball. Such mechanisms, requiring multiple components, are expensive to manufacture and prone to failure.

What is needed, therefore, is a modular screw system that overcomes one or more of the problems in the prior art and that also allows for modular assembly of the tulip and screw components at the time of surgery, prior to implantation. Such a system would ideally be simple and reliable, without additional components which would defeat any cost saving benefit.

### SUMMARY OF THE INVENTION

Providing an unassembled modular pedicle screw system to the surgeon has several important benefits. Prior art systems have large selections of screw shaft types and sizes, each one with a permanently mounted tulip component. A modular system, however, can offer relatively few tulip components which can be assembled to the desired screw components at the time of surgery. This significantly reduces inventory costs, as the tulip components are typically the most expensive component of the system. Additionally, it is possible to offer a wider array of screw sizes and styles, without having to increase the number of tulip components in an individual surgical kit.

It is, therefore, an object of an embodiment of the invention to provide an improved modular multi-axial screw system.

Another object of an embodiment of the invention is to provide a screw system that can be assembled at the time of surgery by a surgeon.

Still another object of the invention is to provide a tulip or receiver component that is adapted and capable of being used with a plurality of screws having different shaft types and sizes.

Still another object of the invention is to provide a screw system having a tulip or receiver that allows for modular assembly of the tulip and receiver and the screw component at the time of surgery.

In one aspect, one embodiment of the invention comprises a modular pedicle screw comprising a tulip or receiver having a body comprising a through bore for receiving a screw having a screw head, the tulip or receiver also comprising a rod receiving channel for receiving a rod, the body comprising a seat for receiving and supporting at least a portion of the screw head, a wall having at least one resilient or elastic arm, the at least one resilient or elastic arm having at least a portion that is operatively positioned in relation to the screw head to retain the screw head in the tulip or receiver.

In another aspect, another embodiment of the invention a tulip or receiver comprising a body comprising a through bore for receiving a screw having a screw head, a rod receiving channel for receiving a rod, a seat for receiving and supporting at least a portion of the screw head, a wall having at least one resilient or elastic arm, the at least one resilient or elastic arm having at least a portion that is operatively positioned in relation to the screw head to retain the screw head in the tulip or receiver.

These and other objects and advantages of the invention will be apparent from the following description, the accompanying drawings and the appended claims.

### BRIEF DESCRIPTION OF THE ACCOMPANYING DRAWINGS

FIG. 1 is a perspective view of one embodiment showing a modular receiver and tulip, screw, rod and securing cap;

FIG. 2 is a view of the embodiment shown in FIG. 1 showing at least one generally elastic or resilient arm integrally or monolithically formed in a side wall of the tulip or receiver;

FIG. 3 is a perspective view showing various details of the tulip or receiver;

FIG. 4 is another perspective view showing further details of the tulip or receiver including the seat for supporting a screw head of the screw;

FIG. 5 is a sectional view showing detail of at least one or a plurality of the elastic or resilient arms;

FIG. 6 is a fragmentary view illustrating the operative relationship of a detent end of the at least one or a plurality of elastic or resilient arms in association with a surface of a screw head;

FIG. 7 is a fragmentary view of the screw in accordance with one embodiment of the invention;

FIG. 8 is a sectional view similar to the view shown in FIG. 5 illustrating a rod situated in the tulip or receiver;

FIG. 9 is another sectional view similar to FIG. 8 illustrating various details of the tulip or receiver after it has received a cap;

FIG. 10 is a sectional view illustrating the relationship of the screw relative to a bore of the tulip or receiver when the screw is inserted therethrough;

FIG. 11 illustrates the screw being inserted into the tulip or receiver;

FIGS. 12A-12C illustrate the screw as it is inserted into the tulip or receiver and the response of the at least one or a plurality of elastic or resilient arms being deflected in response to the movement of the screw head into the tulip or receiver;

FIG. 13 is a view similar to FIG. 12C showing further details;

FIG. 14 illustrates an embodiment wherein the tulip or receiver comprises a plurality of arms in at least one or a plurality of the generally opposing walls of the tulip or receiver, with the plurality of arms each having a longitudinal axis that is generally parallel to an axis of the tulip or receiver; and

FIGS. 15 and 16 illustrate at least one or a plurality of elastic or resilient arms integrally formed in the tulip or receiver and having an axis that is generally transverse or non-parallel with respect to an axis of the tulip or receiver.

### DETAILED DESCRIPTION OF THE PREFERRED EMBODIMENTS

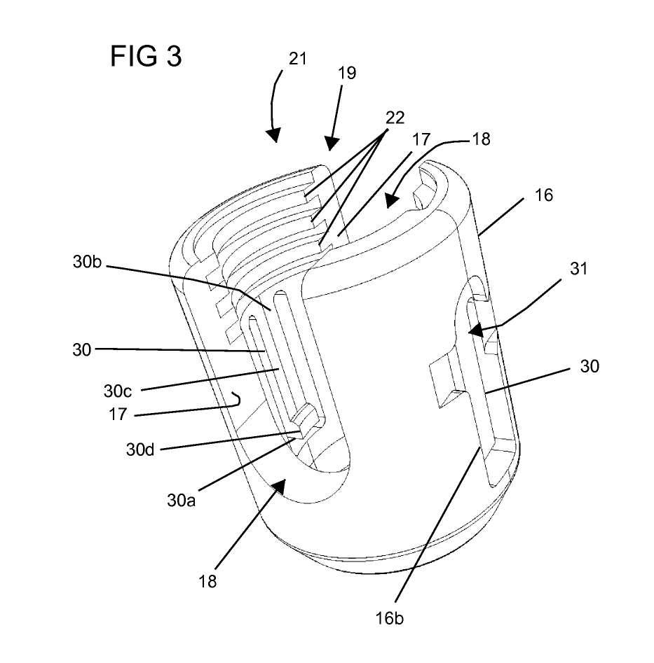

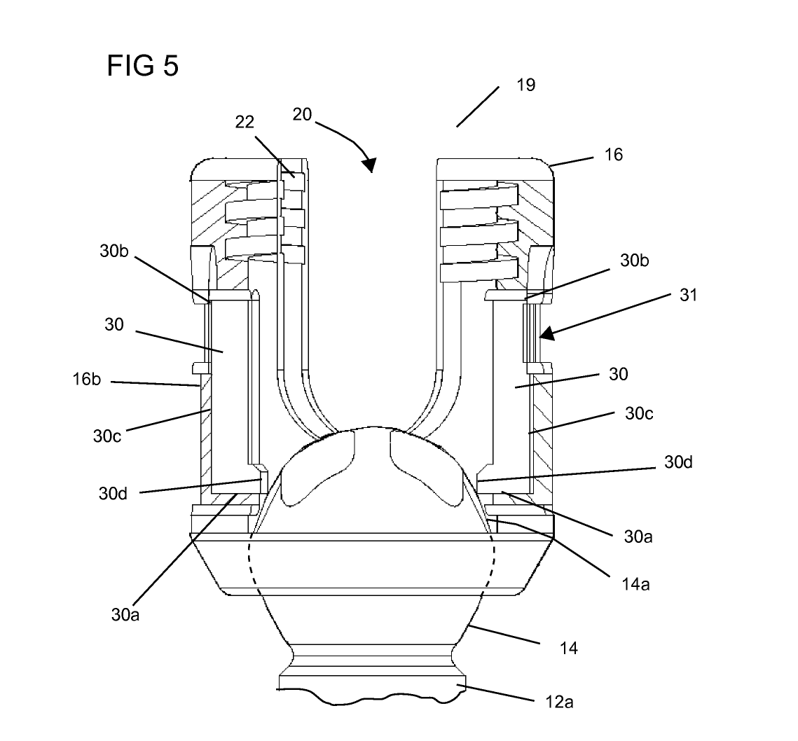

A multi-axial or polyaxial screw system or modular screw assembly 10 is shown. The multi-axial or polyaxial screw or modular screw assembly 10 comprises a multi-axial or polyaxial screw 12 having a shank 12a and a screw head or ball 14 (FIG. 5). The multi-axial or polyaxial screw or modular screw assembly 10 further comprises a tulip or receiver 16 having a pair of generally opposing and generally U-shaped walls 17 that define a plurality of generally U-shaped channels 18 (FIG. 3), respectively, that cooperate to define a rod-receiving area 20 for receiving a rod 32.

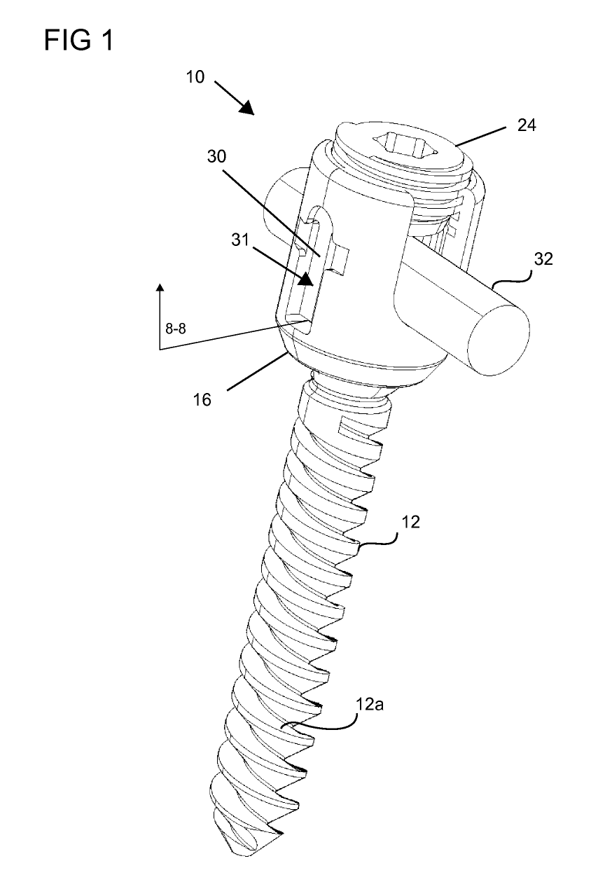

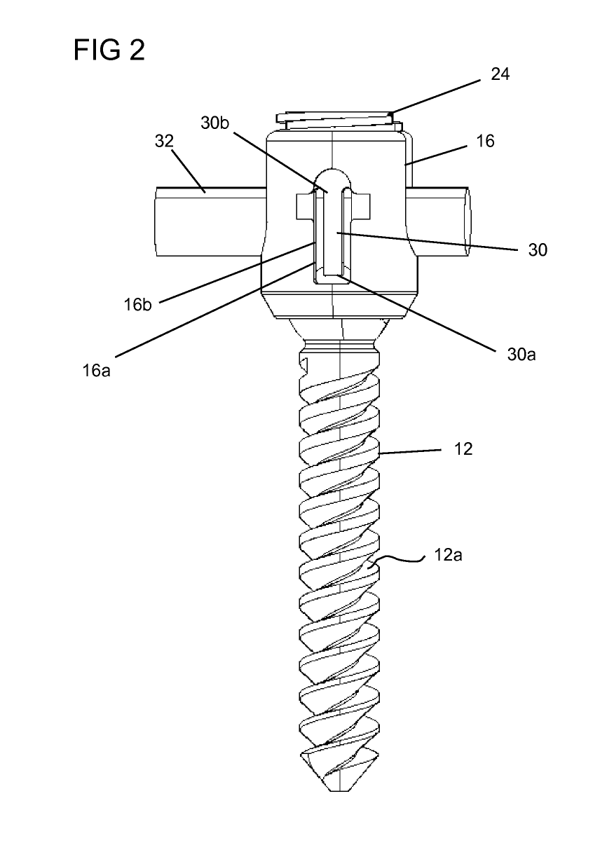

The tulip or receiver 16 further comprises a plurality of threads 22 that define female threads for receiving a threaded cap 24 (FIGS. 1 and 2). Although not shown, the lock or means for securing the rod 32 in the tulip or receiver 16 could be the capless locking system as shown and described in U.S. Pat. Nos. 7,604,655; 7,662,172; 7,686,835; 7,717,943; 8,012,185; 8,016,866; 8,066,745; 8,092,504; 8,097,025; 8,142,481; 8,147,522; 8,382,806; and U.S. Publication Nos. 2007/0123867 and 2013/0150893. These patents and applications are incorporated herein by reference and made a part hereof.

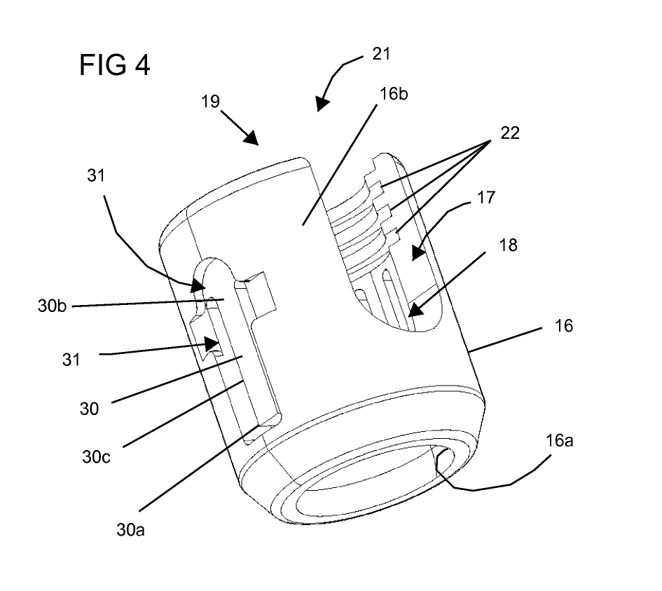

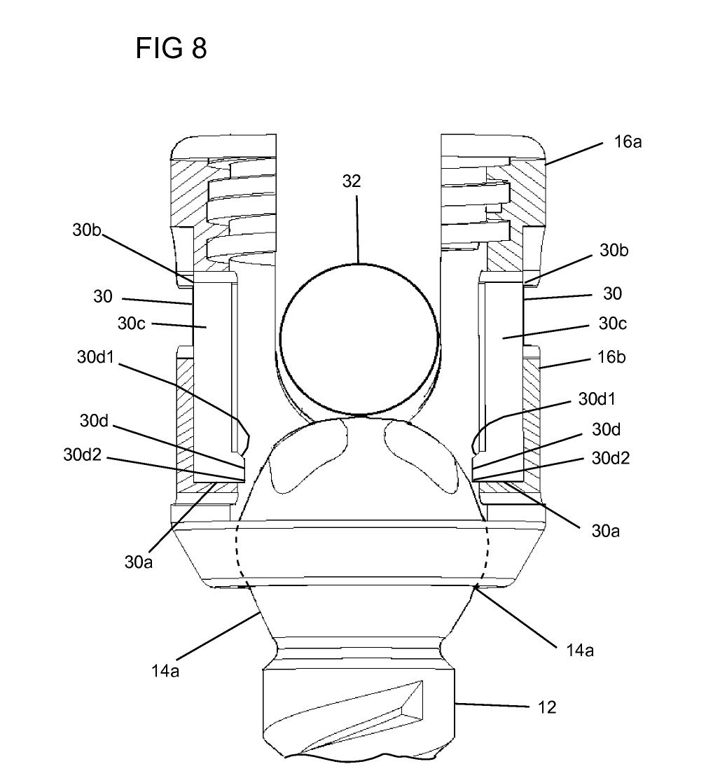

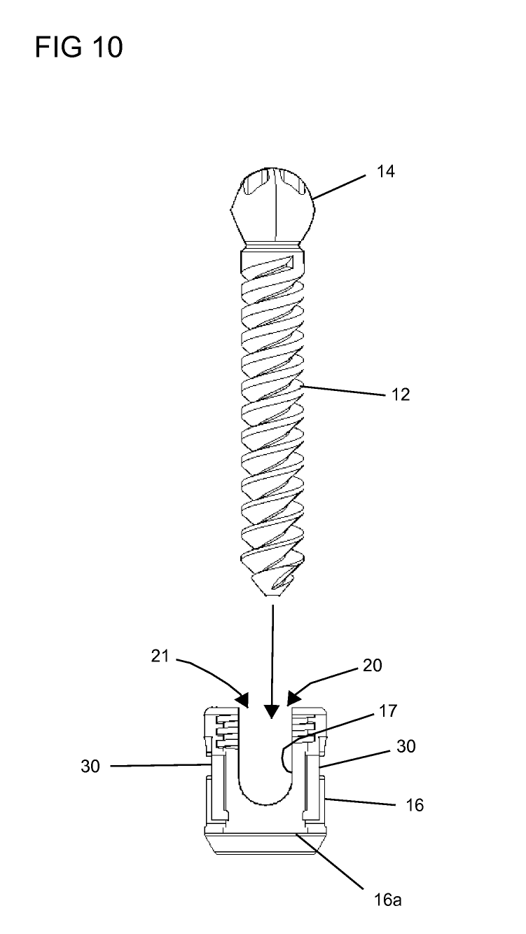

The rod-receiving area 20 (FIG. 5) is adapted to receive the rod 32 (FIGS. 1 and 8) as shown. The tulip or receiver 16 receives the threaded cap 24 which, when screwed, forces the rod 32 against the screw head or ball 14 of the multi-axial or polyaxial screw 12 which in turn forces a lower spherical portion 14a (FIG. 5) of the screw head or ball 14 into engagement with and against a tulip seat 16a (FIGS. 4 and 10) of the tulip or receiver 16 as best illustrated in FIGS. 1, 2, 4 and 8-10.

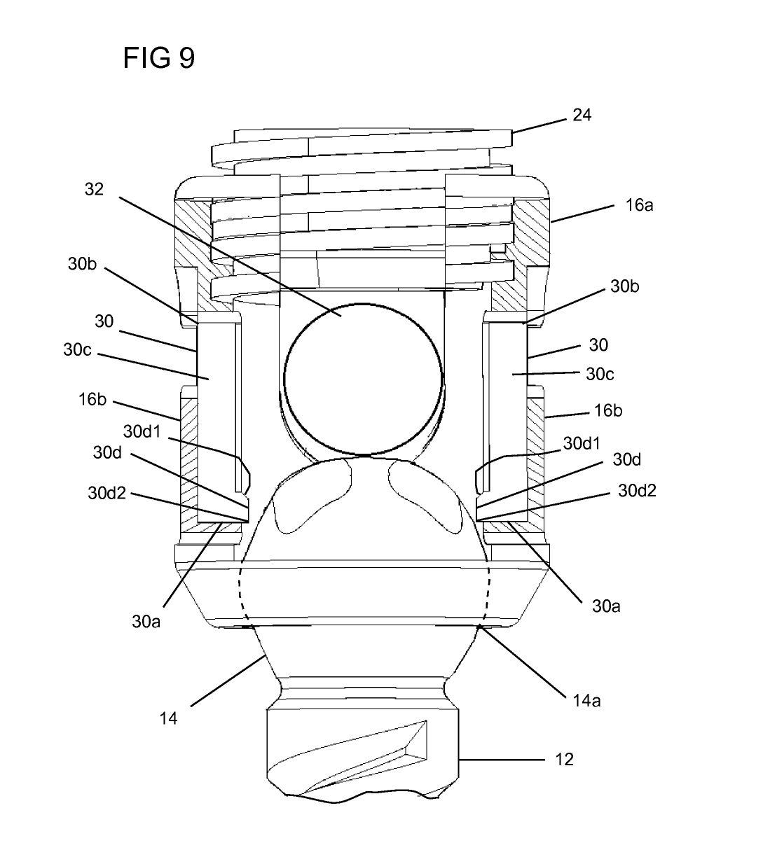

In the embodiment being described, the tulip or receiver 16 of the multi-axial or polyaxial screw 12 is modified with at least one or a plurality of integral, monolithically formed resilient or elastic members or arms 30 which allow for the simple and reversible assembly and retention of the screw head or ball 14 into the tulip seat 16a of the tulip or receiver 16. In the illustration, the tulip or receiver 16 is machined to have cut-out areas 31 (FIGS. 3 and 4) that define the at least one or plurality of resilient members or arms 30. In a preferred embodiment, each of the at least one or a plurality of integral, monolithically formed resilient or elastic members or arms 30 may be linear or generally linear and may be generally L-shaped in cross-section as illustrated in FIG. 9. As mentioned later herein, however, the resilient or elastic members or arms 30 may have other shapes such as an arcuate or curved shape and could have other orientations relative to the axis of the tulip or receiver 16.

As mentioned, each of the at least one or plurality of arms 30 may be generally linear or may have a curved or arcuate shape is desired. Each of the at least one or plurality of arms 30 have a distal end 30a and a proximally attached or integral end 30b that is proximate to a rod-receiving end 19 (FIG. 5) of the tulip or receiver 16. An intermediate elongated portion 30c joins the integral end 30b and distal end 30a. Although the at least one or plurality of arms 30 are shown to be integral or monolithically formed in the tulip or receiver 16, they could be non-integral and/or non-monolithic separate components adhered or affixed to the tulip or receiver 16, for example, by a fastener such as a screw.

Each of the at least one or plurality of arms 30 is located in a side wall 16b (FIGS. 1-4) of the tulip or receiver 16 and is dimensioned to avoid interference with the locking aspects of the rod 32, threaded cap 24, or screw head or ball 14 rotation. The distal end 30a comprises a projection portion or detent 30d (FIG. 8) that may optionally have a beveled edge 30d1.

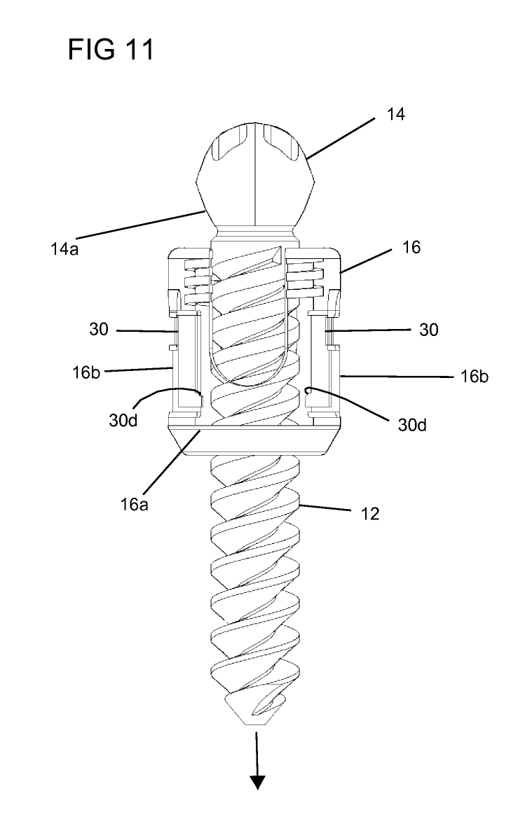

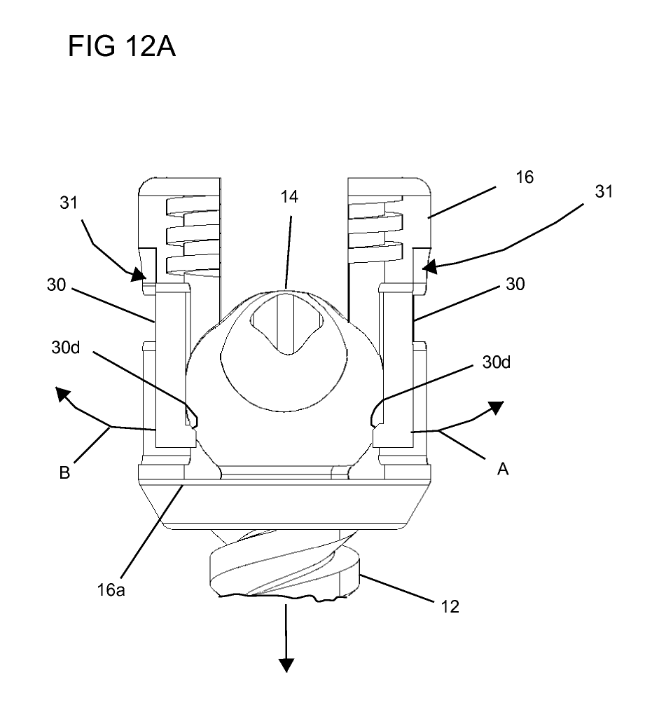

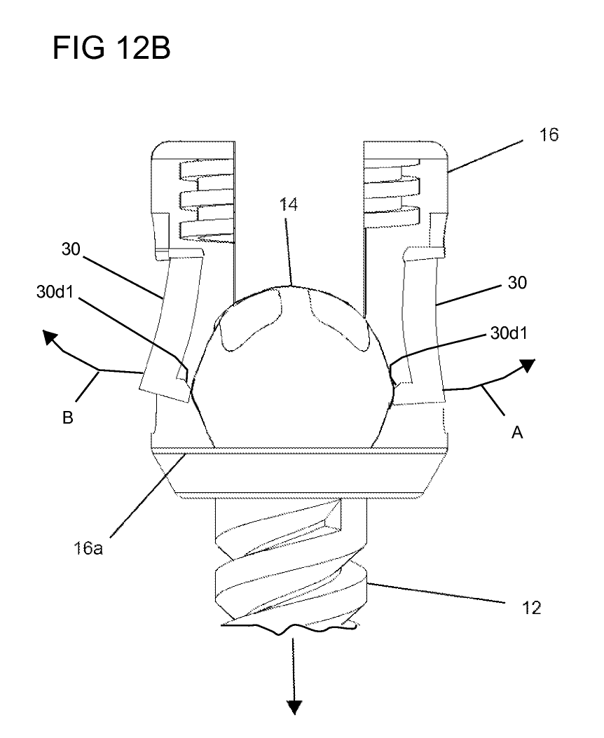

As best illustrated in FIGS. 10-14, the multi-axial or polyaxial screw 12 is inserted axially into a bore 21 of the tulip or receiver 16 and driven downward (as viewed in FIG. 10) into the tulip or receiver 16, as illustrated in FIGS. 10-12C, until the screw head or ball 14 engages the detents 30d and/or beveled surface 30d2 and deflects the at least one or plurality of arms 30 outwardly as indicated by the arrows A and B in FIGS. 12A and 12B. As illustrated in FIGS. 12C and 13, once the major radius of the screw head or ball 14 has cleared the detents 30d, the at least one or a plurality of integral, monolithically formed resilient or elastic members or arms 30 return to their home position, as shown in FIGS. 8, 9, 12C and 13. Note that the corner edges 30d1 of detents 30d or a portion of detent 30d may engage the screw head or ball 14 to retain the screw head or ball 14 against the seat 14a as illustrated in FIG. 13 and to further prevent the screw head or ball 14 and multi-axial or polyaxial screw 12 from unscrewing, withdrawing or moving in a direction of arrow C in FIG. 13.

It should be understood that in this position and until the rod 32 and cap 24 are secured in the tulip or receiver 16 the user, such as a surgeon, is free to move or adjust a position of the tulip or receiver 16 on the screw head or ball 14. Note that this can occur both prior to insertion and implantation of the multi-axial or polyaxial screw or modular screw assembly 10 in a patient, but it could also occur in situ.

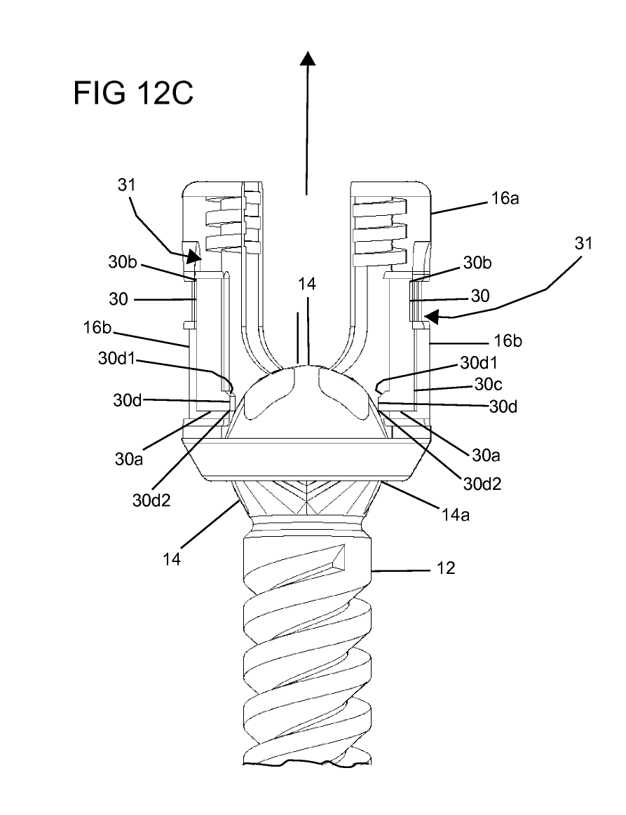

FIGS. 14-16 show additional illustrative embodiments. Like parts are identified with the same part numbers, except a “′” mark has been added. As best illustrated in FIG. 14, note that each of the at least one or plurality of arms 30′ is generally linear and extends in a direction that is generally parallel to an axis X (FIG. 14) of the tulip or receiver 16′. It should also be understood that more or fewer at least one or plurality of arms 30′ could be provided as well. In this regard, FIG. 14 shows another embodiment of the invention where the tulip or receiver 16′ comprises a plurality of arms 30′ in each side wall 16b′. In the illustration, the side walls 16b′ each comprise the pair of the plurality of arms 30′.

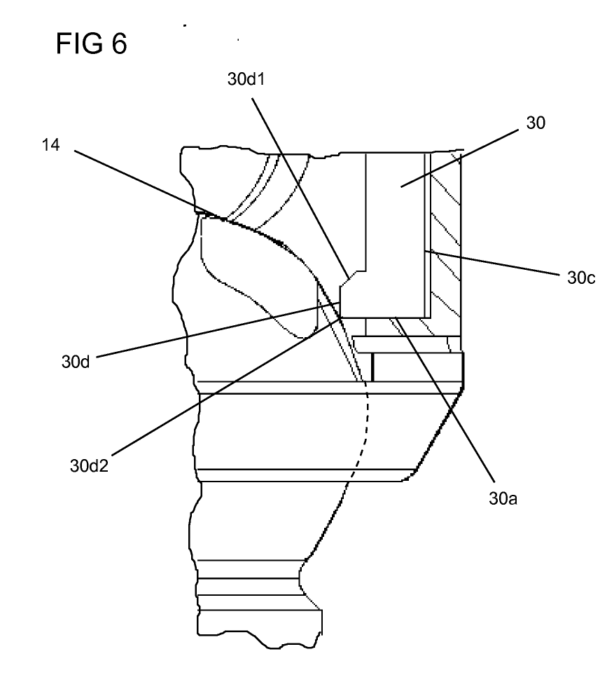

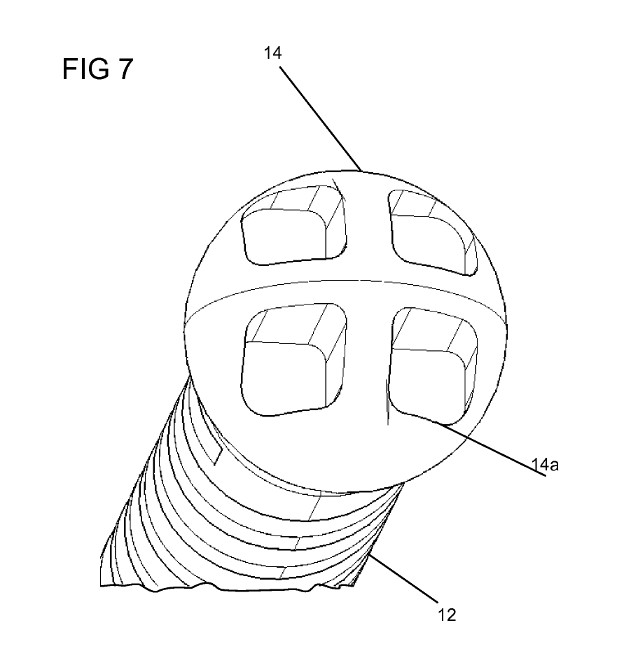

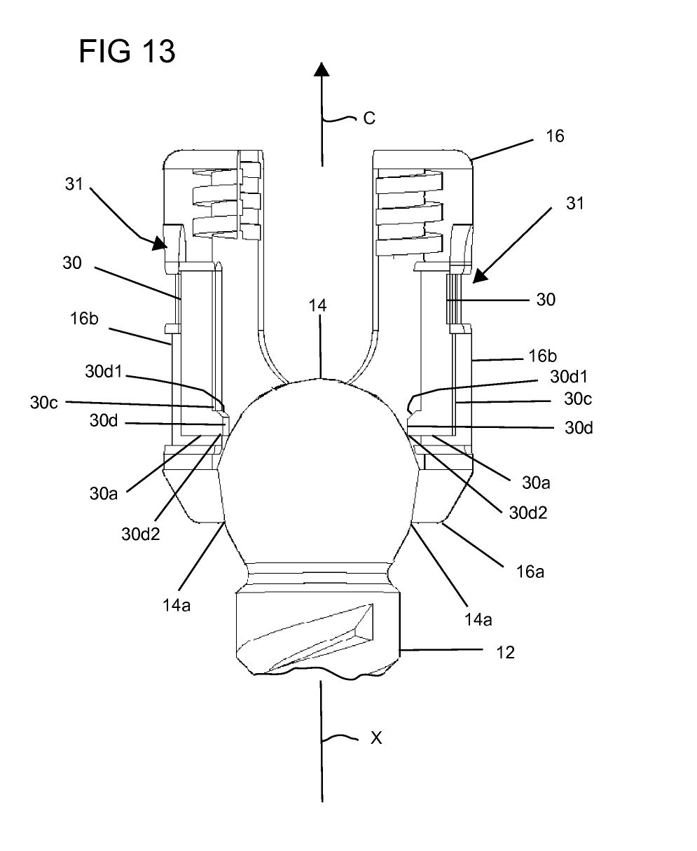

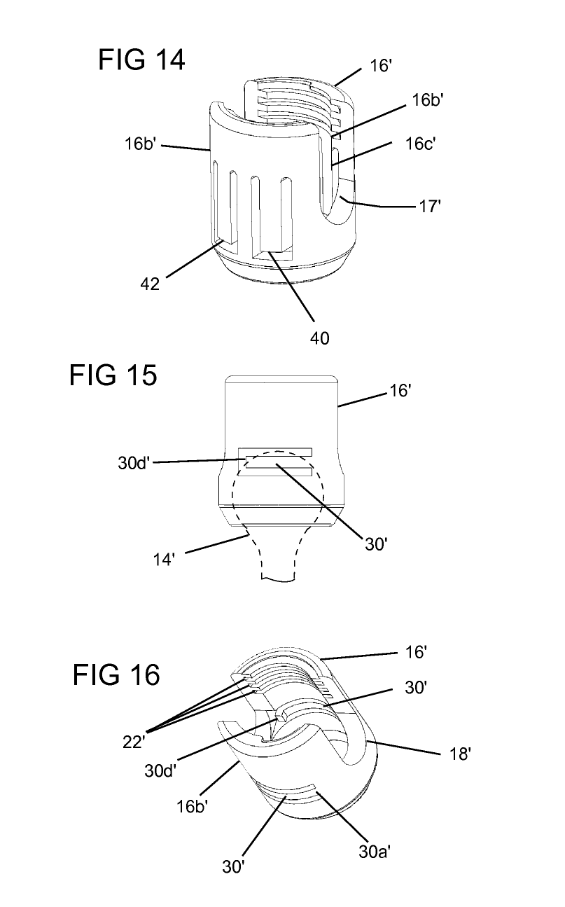

FIGS. 1-14 illustrate the at least one or a plurality of arms 30 having an axis that is generally parallel to the axis of the tulip or receiver 16, but they could take any orientation. For example, FIGS. 15 and 16 show the at least one or a plurality of arms 30′ having a generally curved or arcuate shape and having an axis oriented generally transverse to the axis X as shown. It should be understood, however, that they could take the form of different shapes and be curved, arcuate or non-linear if desired. Moreover, the at least one or a plurality of arms 30′ can take different orientations and have non-linear shapes. For example, FIGS. 15 and 16 illustrate a radial at least one or a plurality of arms 30′ and associated detent end 30d′ having an axis that lies in a generally radial plane and that is generally perpendicular or transverse to the axis X of the tulip or receiver 16′. Note that the radial at least one or a plurality of arms 30′ in this embodiment has an integral end that generally lies in the same radial plane as the detent end 30d′.

Using the embodiments of this invention, a surgeon can assemble the multi-axial or polyaxial screw 12 downward through the tulip or receiver 16, displacing the at least one arm 30, detent 30d and distal end 30a outward, until the screw head or ball 14 radius passes the detent 30d. The detent 30d then returns to its home position and retaining the screw head or ball 14 in the tulip or receiver 16. At this point, the surgeon can implant the multi-axial or polyaxial screw 12 and rod 32 as normal.

Advantageously, the embodiments can also be used with different screws, such as screws having different diameters. The at least one resilient or elastic arm 30 could also comprise an end that is not integral with the tulip or receiver 16.

The invention provides the following advantages:

provides an improved modular multi-axial screw system;

provides a screw system that can be assembled at the time of surgery by a surgeon;

provides a tulip or receiver component that is adapted and capable of being used with a plurality of screws having different shaft types and sizes;

provides a screw system having a tulip or receiver that allows for modular assembly of the tulip and receiver and the screw component at the time of surgery.

While the system, apparatus and method herein described constitute preferred embodiments of this invention, it is to be understood that the invention is not limited to this precise system, apparatus and method, and that changes may be made therein without departing from the scope of the invention which is defined in the appended claims.

What is claimed is:

1. A modular pedicle screw comprising:
a tulip or receiver having a body comprising a through bore for receiving a screw having a screw head, said tulip or receiver also comprising a rod receiving channel for receiving a rod;
said body comprising:
a seat for receiving and supporting at least a portion of said screw head;
a wall having at least one resilient or elastic arm;
said at least one resilient or elastic arm having at least a portion that is operatively positioned in relation to said screw head to retain said screw head in said tulip or receiver.

2. The modular pedicle screw as recited in claim 1 wherein said at least one resilient or elastic arm comprises an end that is integral with said body.

3. The modular pedicle screw as recited in claim 1 wherein said at least one resilient or elastic arm comprises an end that is not integral with said body.

4. The modular pedicle screw as recited in claim 1 wherein said at least one resilient or elastic arm comprises an axis that is generally parallel to an axis of said receiver.

5. The modular pedicle screw as recited in claim 1 wherein said at least one resilient or elastic arm comprises an axis that is generally transverse to an axis of said receiver.

6. The modular pedicle screw as recited in claim 1 wherein said at least a portion of said at least one resilient or elastic arm comprises a detent that projects from a longitudinal axis of said at least one resilient or elastic arm.

7. The modular pedicle screw as recited in claim 1 wherein said at least one resilient or elastic arm is generally L-shaped.

8. The modular pedicle screw as recited in claim 6 wherein at least one resilient or elastic arm comprises a first end integral with said receiver and a free end comprising said at least a portion.

9. The modular pedicle screw as recited in claim 8 wherein said detent is located on said free end.

10. The modular pedicle screw as recited in claim 1 wherein said at least one arm is defined by a cut-out area in said body.

11. The modular pedicle screw as recited in claim 1 wherein said body comprises a plurality of arms.

12. The modular pedicle screw as recited in claim 11 wherein each of said plurality of arms each comprise a longitudinal axis that is generally parallel to an axis of said receiver.

13. The modular pedicle screw as recited in claim 11 wherein each of said plurality of arms each comprise a longitudinal axis that is generally transverse to an axis of said receiver.

14. The modular pedicle screw as recited in claim 11 wherein said body comprises a first wall and a generally opposed second wall, said plurality of arms being equally arranged in said first and second walls.

15. The modular pedicle screw as recited in claim 11 wherein said body comprises a first wall and a generally opposed second wall, said plurality of arms being equally arranged in said first and second walls.

16. The modular pedicle screw as recited in claim 11 wherein said body comprises a first wall and a generally opposed second wall, said plurality of arms each having an axis that is generally parallel to an axis of said receiver.

17. The modular pedicle screw as recited in claim 11 wherein said body comprises a first wall and a generally opposed second wall, said plurality of arms each having an axis that is generally transverse to an axis of said receiver.

18. The modular pedicle screw as recited in claim 11 wherein said at least one resilient or elastic arm comprises an end that is integral or monolithic with said body.

19. The modular pedicle screw as recited in claim 11 wherein each of said plurality of arms is generally L-shaped.

20. The modular pedicle screw as recited in claim 19 wherein each of said plurality of arms is defined by a cut-out in said body.

21. The modular pedicle screw as recited in claim 1 wherein said at least one arm is generally linear.

22. The modular pedicle screw as recited in claim 16 wherein said first and second walls lie in a common generally arcuate plan.

23. The modular pedicle screw as recited in claim 1 wherein said at least one arm assumes a home position adapted to cause said at least a portion to engage or cooperate with said screw head to retain it in said receiver.

24. The modular pedicle screw as recited in claim 1 wherein said at least a portion engages said screw head and said at least one arm is deflected away from an axis of said receiver in response to movement of said screw through said through bore so that when said screw engages a seat of said receiver, said at least a portion returns to a retaining position where said at least a portion can engage said screw head to retain said screw in said receiver either before or after a surgical procedure.

25. The modular pedicle screw as recited in claim 1 wherein said at least a portion is urged to a deflected position in response to said screw passing through said bore of said receiver and said screw head engaging said at least a portion and driving it radially away from an axis of the receiver in response to movement of said screw.

26. The modular pedicle screw as recited in claim 1 wherein said body comprises a female thread, said screw further comprising a cap for mating with said thread and for securing and locking said rod in said receiver.

27. The modular pedicle screw as recited in claim 1 wherein said screw is selected from a group of screws having different diameters.

28. A tulip or receiver comprising:
a body comprising:
a through bore for receiving a screw having a screw head:
a rod receiving channel for receiving a rod:
a seat for receiving and supporting at least a portion of said screw head;
a wall having at least one resilient or elastic arm, said at least one resilient or elastic arm having at least a portion that is operatively positioned in relation to said screw head to retain said screw head in said tulip or receiver.

29. The tulip or receiver as recited in claim 28 wherein said at least one resilient or elastic arm comprises an end that is integral with said body.

30. The tulip or receiver as recited in claim 28 wherein said at least one resilient or elastic arm comprises an end that is not integral with said body.

31. The tulip or receiver as recited in claim 28 wherein said at least one resilient or elastic arm comprises an axis that is generally parallel to an axis of said receiver.

32. The tulip or receiver as recited in claim 28 wherein said at least one resilient or elastic arm comprises an axis that is generally transverse to an axis of said receiver.

33. The tulip or receiver as recited in claim 28 wherein said at least a portion of said at least one resilient or elastic arm comprises a detent that projects from a longitudinal axis of said at least one resilient or elastic arm.

34. The tulip or receiver as recited in claim 28 wherein said at least one resilient or elastic arm is generally L-shaped.

35. The tulip or receiver as recited in claim 33 wherein at least one resilient or elastic arm comprises a first end integral with said receiver and a free end comprising said at least a portion.

36. The tulip or receiver as recited in claim 35 wherein said detent is located on said free end.

37. The tulip or receiver as recited in claim 28 wherein said at least one arm is defined by a cut-out area in said body.

38. The tulip or receiver as recited in claim 28 wherein said body comprises a plurality of arms.

39. The tulip or receiver as recited in claim 38 wherein each of said plurality of arms each comprise a longitudinal axis that is generally parallel to an axis of said receiver.

40. The tulip or receiver as recited in claim 38 wherein each of said plurality of arms each comprise a longitudinal axis that is generally transverse to an axis of said receiver.

41. The tulip or receiver as recited in claim 38 wherein said body comprises a first wall and a generally opposed second wall, said plurality of arms being equally arranged in said first and second walls.

42. The tulip or receiver as recited in claim 38 wherein said body comprises a first wall and a generally opposed second wall, said plurality of arms being equally arranged in said first and second walls.

43. The tulip or receiver as recited in claim 38 wherein said body comprises a first wall and a generally opposed second wall, said plurality of arms each having an axis that is generally parallel to an axis of said receiver.

44. The tulip or receiver as recited in claim 38 wherein said body comprises a first wall and a generally opposed second wall, said plurality of arms each having an axis that is generally transverse to an axis of said receiver.

45. The tulip or receiver as recited in claim 38 wherein said at least one resilient or elastic arm comprises an end that is integral or monolithic with said body.

46. The tulip or receiver as recited in claim 38 wherein each of said plurality of arms is generally L-shaped.

47. The tulip or receiver as recited in claim 37 wherein said at least one arm is defined by a cut-out in said body.

48. The tulip or receiver as recited in claim 28 wherein said at least one arm is generally linear.

49. The tulip or receiver as recited in claim 28 wherein said at least one arm is curved or arcuate.

50. The tulip or receiver as recited in claim 43 wherein said first and second walls lie in a common generally arcuate plan.

51. The tulip or receiver as recited in claim 28 wherein said at least one arm assumes a home position adapted to cause said at least a portion to engage or cooperate with said screw head to retain it in said receiver.

52. The tulip or receiver as recited in claim 28 wherein said at least a portion engages said screw head and said at least one arm is deflected away from an axis of said receiver in response to movement of said screw through said through bore so that when said screw engages a seat of said receiver, said at least a portion returns to a retaining position where said at least a portion can engage said screw head to retain said screw in said receiver either before or after a surgical procedure.

53. The tulip or receiver as recited in claim 28 wherein said at least a portion is urged to a deflected position in response to said screw passing through said bore of said receiver and said screw head engaging said at least a portion and driving it radially away from an axis of the receiver in response to movement of said screw.

54. The tulip or receiver as recited in claim 28 wherein said body comprises a female thread, said tulip or receiver further comprising a cap for mating with said thread and for securing and locking said rod in said receiver.
